(19) United States
(12) Patent Application Publication
(10) Pub. No.: US 2018/0008670 A1
(43) Pub. Date: Jan. 11, 2018

(54) CHIMERIC ANTIGEN RECEPTOR TARGETING OF TUMOR ENDOTHELIUM

(71) Applicant: Batu Biologics, Inc., San Diego, CA (US)

(72) Inventors: Samuel C. Wagner, San Diego, CA (US); Thomas E. Ichim, San Diego, CA (US); Boris Minev, San Diego, CA (US)

(73) Assignee: Batu Biologics, Inc., San Diego, CA (US)

(21) Appl. No.: 15/712,887

(22) Filed: Sep. 22, 2017

Related U.S. Application Data
(63) Continuation of application No. 15/018,797, filed on Feb. 8, 2016, now abandoned.
(60) Provisional application No. 62/112,999, filed on Feb. 6, 2015.

(51) Int. Cl. A61K 38/17; C07K 14/705; C07K 14/725

(52) U.S. Cl. CPC A61K 38/1793; A61K 38/1774; C07K 14/7051; C07K 14/70521; C07K 14/70578; A61K 35/17; C07K 2319/03; C07K 2319/33; C07K 2319/70; C07K 2319/74; C07K 2319/02

(57) ABSTRACT

Disclosed are methods, protocols, and compositions of matter related to utilization of chimeric antigen receptor (CAR) expressing cells for the targeting of tumor endothelium utilizing chimeric antigen receptor expressing stem cells. In one embodiment tumor endothelium specific antigens are utilized as targets of the antigen binding domain of a CAR, which is attached to an extracellular hinge domain, a domain that transverses the T cell membrane and an intracellular domain associated with T cell signaling. Suitable antigens for the practice of the invention include TEM-1, ROBO-4, surviving, and FasL. In other aspects of the invention antigens are identified through serological analysis of recombinant cDNA expression libraries (SEREX) using plasma from a patient immunized with placental endothelial cells.

### CROSS-REFERENCE TO RELATED APPLICATION

This application claims the benefit of and priority to U.S. Non-Provisional patent application Ser. No. 15/018,797 filed on Feb. 8, 2016, which claims the benefit of U.S. Provisional Application No. 62/112,999 filed on Feb. 6, 2015, the contents of which are incorporated herein by reference in its entirety.

### BACKGROUND OF THE INVENTION

The standard of treatments for cancer are surgery, radiation therapy, and chemotherapy. Unfortunately, these approaches are often not curative and are associated with extremely high toxicity and adverse effects. Immunotherapy which uses the body's immune system, either directly or indirectly, to shrink or eradicate cancer has been studied for many years as an adjunct to conventional cancer therapy. It is believed that the human immune system is an untapped resource for cancer therapy and that effective treatment can be developed once the components of the immune system are properly harnessed. As key immunoregulatory molecules and signals of immunity are identified and prepared as therapeutic reagents, the clinical effectiveness of such reagents can be tested using established cancer models. Immunotherapeutic strategies include administration of vaccines, activated cells, antibodies, cytokines, chemokines, as well as small molecular inhibitors, anti-sense oligonucleotides, and gene therapy. It is believed by many that immunotherapy offers the potential for treatment of cancer without the toxicities associated with current approaches to cancer therapy.

Unfortunately while numerous studies have demonstrated that immune cells are capable of killing cancers in vitro or at a small scale in vivo, the power of immunotherapy has not been fully utilized due to: a) lack of ability to expand immunological cells capable of specifically killing tumors; and b) tumor initiated defense mechanisms.

Chimeric antigen receptor (CAR) T cells overcome some of these limitations. CAR T cells do not need MHC I presentation of antigen since they usually have an antibody domain connected to T cell receptor (TCR) signaling molecules. Accordingly, CAR T cells are not limited by need for MHC antigen presentation. This is important since many tumors downregulate MHC or associated antigen processing machinery such as TAP.

Unfortunately limitations of CAR T cells include the lack of ability for the T cells to infiltrate deep into tumor tissue. The current invention overcomes this by utilizing CAR T cells to stimulate immunity towards tumor endothelium. Since tumor endothelium is in direct contact with the blood, the ability of CART cells to destroy the tumor through abrogation of its blood supply.

### DETAILED DESCRIPTION OF THE INVENTION

Unless defined differently, all technical and scientific terms used herein have the same meanings as commonly understood by one of skill in the art to which the disclosed invention belongs. In particular, the following terms and phrases have the following meaning.

“Treating a cancer”, “inhibiting cancer”, “reducing cancer growth” refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells.

The terms “cancer”, “cancer cell”, “tumor”, and “tumor cell” are used interchangeably herein and refer generally to a group of diseases characterized by uncontrolled, abnormal growth of cells (e.g., a neoplasioa). In some forms of cancer, the cancer cells can spread locally or through the bloodstream and lymphatic system to other parts of the body (“metastatic cancer”).

“Ex vivo activated lymphocytes”, “lymphocytes with enhanced antitumor activity” and “dendritic cell cytokine induced killers” are terms used interchangeably to refer to composition of cells that have been activated ex vivo and subsequently reintroduced within the context of the current invention. Although the word “lymphocyte” is used, this also includes heterogenous cells that have been expanded during the ex vivo culturing process including dendritic cells, NKT cells, gamma delta T cells, and various other innate and adaptive immune cells.

As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma.

The term “leukemia” is meant broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemi.

The term “carcinoma” refers to a malignant new growth made up of epithelial cells tending to infiltrate the surrounding tissues, and/or resist physiological and non-physiological cell death signals and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti.

The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some particular embodiments of the invention, the cancer treated is a melanoma. The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma. The term “polypeptide” is used interchangeably with “peptide”, “altered peptide ligand”, and “flourocarbonated peptides.”

The term “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

The term “T cell” is also referred to as T lymphocyte, and means a cell derived from thymus among lymphocytes involved in an immune response. The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an αβ T cell expressing TCR αβ chains, and a γδ T cell expressing TCR γ and δ chains. The T cell includes a precursor cell of a T cell in which differentiation into a T cell is directed. Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T cells and derived from hematopoietic cells can be used in the present invention. These cells may have been activated by cytokine such as IL-2 in vivo or ex vivo. As these cells, any of cells collected from a living body, or cells obtained via ex vivo culture, for example, a T cell population obtained by the method of the present invention as it is, or obtained by freeze preservation, can be used.

The term “antibody” is meant to include both intact molecules as well as fragments thereof that include the antigen-binding site. Whole antibody structure is often given as H2L and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity. The variable regions of either H or L chains contain the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure. The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain.

The term “effective amount” or “therapeutically effective amount” means a dosage sufficient to treat, inhibit, or alleviate one or more symptoms of a disease state being treated or to otherwise provide a desired pharmacologic and/or physiologic effect, especially enhancing T cell response to a selected antigen. The precise dosage will vary according to a variety of factors such as subject-dependent variables (e.g., age, immune system health, etc.), the disease, and the treatment being administered.

The terms “individual”, “host”, “subject”, and “patient” are used interchangeably herein, and refer to a mammal, including, but not limited to, primates, for example, human beings, as well as rodents, such as mice and rats, and other laboratory animals.

As used herein, the term “treatment regimen” refers to a treatment of a disease or a method for achieving a desired physiological change, such as increased or decreased response of the immune system to an antigen or immunogen, such as an increase or decrease in the number or activity of one or more cells, or cell types, that are involved in such response, wherein said treatment or method comprises administering to an animal, such as a mammal, especially a human being, a sufficient amount of two or more chemical agents or components of said regimen to effectively treat a disease or to produce said physiological change, wherein said chemical agents or components are administered together, such as part of the same composition, or administered separately and independently at the same time or at different times (i.e., administration of each agent or component is separated by a finite period of time from one or more of the agents or components) and where administration of said one or more agents or components achieves a result greater than that of any of said agents or components when administered alone or in isolation.

The term “anergy” and “unresponsiveness” includes unresponsiveness to an immune cell to stimulation, for example, stimulation by an activation receptor or cytokine. The anergy may occur due to, for example, exposure to an immune suppressor or exposure to an antigen in a high dose. Such anergy is generally antigen-specific, and continues even after completion of exposure to a tolerized antigen. For example, the anergy in a T cell and/or NK cell is characterized by failure of production of cytokine, for example, interleukin (IL)-2. The T cell anergy and/or NK cell anergy occurs in part when a first signal (signal via TCR or CD-3) is received in the absence of a second signal (costimulatory signal) upon exposure of a T cell and/or NK cell to an antigen.

The term “enhanced function of a T cell”, “enhanced cytotoxicity” and “augmented activity” means that the effector function of the T cell and/or NK cell is improved. The enhanced function of the T cell and/or NK cell, which does not limit the present invention, includes an improvement in the proliferation rate of the T cell and/or NK cell, an increase in the production amount of cytokine, or an improvement in cytotoxity. Further, the enhanced function of the T cell and/or NK cell includes cancellation and suppression of tolerance of the T cell and/or NK cell in the suppressed state such as the anergy (unresponsive) state, or the rest state, that is, transfer of the T cell and/or NK cell from the suppressed state into the state where the T cell and/or NK cell responds to stimulation from the outside.

The term “expression” means generation of mRNA by transcription from nucleic acids such as genes, polynucleotides, and oligonucleotides, or generation of a protein or a polypeptide by transcription from mRNA. Expression may be detected by means including RT-PCR, Northern Blot, or in situ hybridization.

“Suppression of expression” refers to a decrease of a transcription product or a translation product in a significant amount as compared with the case of no suppression. The suppression of expression herein shows, for example, a decrease of a transcription product or a translation product in an amount of 30% or more, preferably 50% or more, more preferably 70% or more, and further preferably 90% or more.

The invention discloses compositions and methods for treating through the generation of an immune response to blood vessels that are preferentially associated with tumors. The immunogeneicity of tumor blood vessels as a vaccination target has been demonstrated previously.

Zuange et al. described the induction of tumor endothelial specific immunity through the immunization against ROBO4. Mice were immunised with the extracellular domain of mouse Robo4, fused to the Fc domain of human immunoglobulin within an adjuvant. Vaccinated mice demonstrated a potent antibody response to Robo4, with no objectively detectable adverse effects on healthy angiogenesis including menstruation or wound healing. Robo4 vaccinated mice showed impaired fibrovascular invasion and angiogenesis in a rodent sponge implantation assay, as well as a reduced growth of implanted syngeneic Lewis lung carcinoma. The ability of the vaccine to inhibit angiogenesis in this lung cancer model was demonstrated to be dependent on the humoral arm of the immune system but not on the cytotoxic arm. Specifically, it was demonstrated that deletion of antibody generating activity negated antitumor activity but that depletion of the cytotoxic arm of the immune system (CD8 T Cells) allowed for maintenance of antitumor activity.

Additionally, the authors demonstrated that an adjuvant free soluble Robo4-carrier conjugate can retard tumor growth in carrier primed mice [1]. Accordingly in one embodiment of the invention CAR-T cells are generated with specificity towards ROBO-4. Numerous means of generating CAR-T cells are known in the art. In one embodiment of the invention FMC63-28z CAR (Genebank identifier HM852952.1), is used as the template for the CAR except the anti-CD19, single-chain variable fragment sequence is replaced with an ROBO-4 fragment. The construct is synthesized and inserted into a pLNCX retroviral vector. Retroviruses encoding the ROBO-4-specific CAR are generated using the retrovirus packaging kit, Ampho (Takara), following the manufacturer's protocol. For generation of CAR-T cells donor blood is obtained and after centrifugation on Ficoll-Hypaque density gradients (Sigma-Aldrich), PBMCs are plated at 2×10(6) cells/mL in cell culture for 2 hours and the non-adherent cells are collected. The cells were then stimulated for 2 days on a non-tissue-culture-treated 24-well plate coated with 1 μg/mL OKT3 (Biolegend) at 1×10(6) cells/mL and in the presence of 1 μg/mL of anti-human CD28 antibody (Biolegend).

For retrovirus transduction, a 24-well plate are coated with RetroNectin (Takara) at 4° C. overnight, according to the manufacturer's protocol, and then blocked with 2% BSA at room temperature for 30 min. The plate was then loaded with retrovirus supernatants at 300 μL/well and incubated at 37° C. for 6 h. Next, 1×10(6) stimulated PBLs in 1 mL of medium are added to 1 mL of retrovirus supernatants before being transferred to the pre-coated wells and cultured at 37° C. for 2 d. The cells are then transferred to a tissue-culture-treated plate at 1×10(6) cells/mL and cultured in the presence of 100 U/mL of recombinant human IL-2 [2].

Other means of generating CARs are known in the art and incorporated by reference. For example, Groner's group genetically modified T lymphocytes and endowed them with the ability to specifically recognize cancer cells. Tumor cells overexpressing the ErbB-2 receptor served as a model. The target cell recognition specificity was conferred to T lymphocytes by transduction of a chimeric gene encoding the zeta-chain of the TCR and a single chain antibody (scFv(FRP5)) directed against the human ErbB-2 receptor. The chimeric scFv(FRP5)-zeta gene was introduced into primary mouse T lymphocytes via retroviral gene transfer. Naive T lymphocytes were activated and infected by cocultivation with a retrovirus-producing packaging cell line. The scFv(FRP5)-zeta fusion gene was expressed in >75% of the T cells. These T cells lysed ErbB-2-expressing target cells in vitro with high specificity. In a syngeneic mouse model, mice were treated with autologous, transduced T cells. The adoptively transferred scFv(FRP5)-zeta-expressing T cells caused total regression of ErbB-2-expressing tumors. The presence of the transduced T lymphocytes in the tumor tissue was monitored. No humoral response directed against the transduced T cells was observed. Abs directed against the ErbB-2 receptor were detected upon tumor lysis [3].

Hombach et al. constructed an anti-CEA chimeric receptor whose extracellular moiety is composed of a humanized scFv derived from the anti-CEA mAb BW431/26 and the CH2/CH3 constant domains of human IgG. The intracellular moiety consists of the gamma-signaling chain of the human Fc epsilon RI receptor constituting a completely humanized chimeric receptor. After transfection, the humBW431/26 scFv-CH2CH3-gamma receptor is expressed as a homodimer on the surface of MD45 T cells. Co-incubation with CEA+ tumor cells specifically activates grafted MD45 T cells indicated by IL-2 secretion and cytolytic activity against CEA+ tumor cells. Notably, the efficacy of receptor-mediated activation is not affected by soluble CEA up to 25 micrograms/ml demonstrating the usefulness of this chimeric receptor for specific cellular activation by membrane-bound CEA even in the presence of high concentrations of CEA, as found in patients during progression of the disease [4]. These methods are described to guide one of skill in the art to practicing the invention, which in one embodiment is the utilization of CAR T cell approaches towards targeting tumor endothelium as comparted to simply targeting the tumor itself.

Targeting of mucins associated with cancers has been performed with CAR T cells by grafting the antibody that binds to the mucin with CD3 zeta chain. In an older publication chimeric immune receptor consisting of an extracellular antigen-binding domain derived from the CC49 humanized single-chain antibody, linked to the CD3zeta signaling domain of the T cell receptor, was generated (CC49-zeta). This receptor binds to TAG-72, a mucin antigen expressed by most human adenocarcinomas. CC49-zeta was expressed in CD4+ and CD8+ T cells and induced cytokine production on stimulation. Human T cells expressing CC49-zeta recognized and killed tumor cell lines and primary tumor cells expressing TAG-72. CC49-zeta T cells did not mediate bystander killing of TAG-72-negative cells. In addition, CC49-zeta T cells not only killed FasL-positive tumor cells in vitro and in vivo, but also survived in their presence, and were immunoprotective in intraperitoneal and subcutaneous murine tumor xenograft models with TAG-72-positive human tumor cells. Finally, receptor-positive T cells were still effective in killing TAG-72-positive targets in the presence of physiological levels of soluble TAG-72, and did not induce killing of TAG-72-negative cells under the same conditions [5].

For clinical practice of the invention several reports exist in the art that would guide the skilled artisan as to concentrations, cell numbers, and dosing protocols useful. While in the art CAR T cells have been utilized targeting surface tumor antigens, the main issue with this approach is the difficulty of T cells to enter tumors due to features specific to the tumor microenvironment. These include higher interstitial pressure inside the tumor compared to the surroundings [6-19], acidosis inside the tumor [20-40], and expression in the tumor of FasL which kills activated T cells [41-50]. Accordingly the invention seeks to more effectively utilize CAR T cells by directly targeting them to tumor endothelium, which is in direct contact with blood and therefore not susceptible to intratumoral factors the limit efficacy of conventional T cell therapies.

In one embodiment of the invention, protocols similar to Kershaw et al are utilized with the exception that tumor endothelial antigens are targeted as opposed to conventional tumor antigens. Such tumor endothelial antigens include CD93, TEM-1, VEGFR1, and survivin. Antibodies can be made for these proteins, methodologies for which are described in U.S. Pat. Nos. 5,225,539, 5,585,089, 5,693,761, and 5,639,641. In one example that may be utilized as a template for clinical development, T cells with reactivity against the ovarian cancer-associated antigen alpha-folate receptor (FR) were generated by genetic modification of autologous T cells with a chimeric gene incorporating an anti-FR single-chain antibody linked to the signaling domain of the Fc receptor gamma chain. Patients were assigned to one of two cohorts in the study. Eight patients in cohort 1 received a dose escalation of T cells in combination with high-dose interleukin-2, and six patients in cohort 2 received dual-specific T cells (reactive with both FR and allogeneic cells) followed by immunization with allogeneic peripheral blood mononuclear cells. Five patients in cohort 1 experienced some grade 3 to 4 treatment-related toxicity that was probably due to interleukin-2 administration, which could be managed using standard measures. Patients in cohort 2 experienced relatively mild side effects with grade 1 to 2 symptoms. No reduction in tumor burden was seen in any patient. Tracking 111In-labeled adoptively transferred T cells in cohort 1 revealed a lack of specific localization of T cells to tumor except in one patient where some signal was detected in a peritoneal deposit. PCR analysis showed that gene-modified T cells were present in the circulation in large numbers for the first 2 days after transfer, but these quickly declined to be barely detectable 1 month later in most patients [51]. Similar CAR-T clinical studies have been reported for neuroblastoma [52, 53], B cell malignancies [54-66], melanoma [67], ovarian cancer [68], renal cancer [69], mesothelioma [70], and head and neck cancer [71].

In one embodiment of the invention PBMCs are derived from leukapheresis and stimulated with anti-CD3 (OKT3, Ortho Biotech, Raritan, N.J.) and human recombinant IL-2 (600 IU/mL; Chiron, Emeryville, Calif.). After 3 days of culture, ˜5×107 to 1×108 lymphocytes are taken and transduced with retroviral vector supernatant (Cell Genesys, San Francisco, Calif.) encoding the chimeric CAR T recognizing tumor-endothelium specific antigen and subsequently selected for gene integration by culture in G418. In another embodiment the generation of dual-specific T cells is performed, stimulation of T cells is achieved by coculture of patient PBMCs with irradiated (5,000 cGy) allogeneic donor PBMCs from cryopre-served apheresis product (mixed lymphocyte reaction). The MHC haplotype of allogeneic donors is determined before use, and donors that differed in at least four MHC class I alleles from the patient are used. Culture medium consisted of AimV medium (Invitrogen, Carlsbad, Calif.) supplemented with 5% human AB-serum (Valley Biomedical, Winchester, Va.), penicillin (50 units/mL), streptomycin (50 mg/mL; Bio Whittaker, Walkersville, Md.), amphotericin B (Fungizone, 1.25 mg/mL; Biofluids, Rockville, Md.), L-glutamine (2 mmol/L; Mediatech, Herndon, Va.), and human recombinant IL-2 (Proleukin, 300 IU/mL; Chiron). Mixed lymphocyte reaction consisted of 2×106 patient PBMCs and 1×107 allogeneic stimulator PBMCs in 2 mL AimV per well in 24-well plates. Between 24 and 48 wells are cultured per patient for 3 days, at which time transduction is done by aspirating 1.5 mL of medium and replacing with 2.0 mL retroviral supernatant containing 300 IU/mL IL-2, 10 mmol/L HEPES, and 8 μg/mL polybrene (Sigma, St. Louis, Mo.) followed by covering with plastic wrap and centrifugation at 1,000×g for 1 hour at room temperature. After overnight culture at 37° C./5% CO2, transduction is repeated on the following day, and then medium was replaced after another 24 hours. Cells are then resuspended at 1×106/mL in fresh medium containing 0.5 mg/mL G418 (Invitrogen) in 175-cm2 flasks for 5 days before resuspension in media lacking G418. Cells are expanded to 2×109 and then restimulated with allogeneic PBMCs from the same donor to enrich for T cells specific for the donor allogeneic haplotype. Restimulation is done by incubating patient T cells (1×106/mL) and stimulator PBMCs (2×106/mL) in 3-liter Fenwall culture bags in AimV+additives and IL-2 (no G418). Cell numbers were adjusted to 1×106/mL, and IL-2 was added every 2 days, until sufficient numbers for treatment were achieved.

The present invention relates to a strategy of adoptive cell transfer of T cells transduced to express a chimeric antigen receptor (CAR). CARs are molecules that combine antibody-based specificity for a desired antigen (e.g., tumor endothelial antigen) with a T cell receptor-activating intracellular domain to generate a chimeric protein that exhibits a specific anti-tumor endothelium cellular immune activity. In one embodiment the present invention relates generally to the use of T cells genetically modified to stably express a desired CAR that possesses high affinity towards tumor associated endothelium. T cells expressing a CAR are referred to herein as CAR T cells or CAR modified T cells. Preferably, the cell can be genetically modified to stably express an antibody binding domain on its surface, conferring novel antigen specificity that is MHC independent. In some instances, the T cell is genetically modified to stably express a CAR that combines an antigen recognition domain of a specific antibody with an intracellular domain of the CD3-zeta chain or Fc.gamma.RI protein into a single chimeric protein. In one embodiment, the CAR of the invention comprises an extracellular domain having an antigen recognition domain, a transmembrane domain, and a cytoplasmic domain. In one embodiment, the transmembrane domain that naturally is associated with one of the domains in the CAR is used. In another embodiment, the transmembrane domain can be selected or modified by amino acid substitution to avoid binding of such domains to the transmembrane domains of the same or different surface membrane proteins to minimize interactions with other members of the receptor complex. Preferably, the transmembrane domain is the CD8.alpha. hinge domain.

With respect to the cytoplasmic domain, the CAR of the invention can be designed to comprise the CD28 and/or 4-1BB and/or CD40 and/or OX40 signaling domain by itself or be combined with any other desired cytoplasmic domain(s) useful in the context of the CAR of the invention. In one embodiment, the cytoplasmic domain of the CAR can be designed to further comprise the signaling domain of CD3-zeta. For example, the cytoplasmic domain of the CAR can include but is not limited to CD3-zeta, 4-1BB and CD28 signaling modules and combinations thereof. In another embodiment of the invention inhibition of CTLA-4 is performed either by transfection with an shRNA possessing selectively towards CTLA-4 or by constructing the CAR to possess a dominant negative mutant of CTLA-4. This would render the CAR T cell resistant to inhibitory activities of the tumors. Accordingly, the invention provides CAR T cells and methods of their use for adoptive therapy. In one embodiment, the CAR T cells of the invention can be generated by introducing a lentiviral vector comprising a desired CAR, for example a CAR comprising anti-CD19, CD8.alpha. hinge and transmembrane domain, and human 4-1BB and CD3zeta signaling domains, into the cells. The CAR T cells of the invention are able to replicate in vivo resulting in long-term persistence that can lead to sustained tumor control.

1. A method of immunologically inhibiting neoangiogenesis comprising:
a) obtaining a cell population from peripheral blood;
b) transfecting said population with a chimeric antigen receptor (CAR); and
c) introducing said transfected cell population into said patient.

2. The method of claim 1, wherein said blood cell population is selected from a group comprising:
a) peripheral blood mononuclear cells;
b) CD4 T cells;
c) CD8 T cells;
d) NK cells;
e) NKT cells; and
f) gamma delta T cells.

3. The method of claim 2, wherein said CD4 T cells are isolated by means of magnetic separation prior to transfection with CAR.

4. The method of claim 2, wherein said CD8 T cells are isolated by means of magnetic separation prior to transfection with CAR.

5. The method of claim 1, wherein said CAR is comprised of:
a) an antigen binding domain;
b) a transmembrane domain;
c) a costimulatory signaling region; and
d) a CD3 zeta signaling domain.

6. The method of claim 5, wherein said CD3 zeta chain is resistant to cleavage by caspase 3 by means of amino acid substitution.

7. The method of claim 5, wherein the antigen binding domain is an antibody or an antigen-binding fragment thereof.

8. The method of claim 7, wherein the antigen-binding fragment is a Fab or a scFv.

9. The method of claim 5, wherein the antigen binding domain binds to an endothelial cell antigen found preferentially on tumor endothelium.

10. The method of claim 9, wherein said tumor endothelial antigen is selected from a group of antigens comprising:
a) TEM-1;
b) TEM-2;
c) TEM-3;
d) TEM-4;
e) TEM-5;
f) TEM-6;
g) TEM-7;
h) TEM-8;
i) ROBO-4;
j) VEGFR2;
k) CD109;
l) survivin; and
m) CD93.

11. The method of claim 5, wherein said costimulatory signaling region comprises the intracellular domain of a costimulatory molecule selected from the group comprising of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83.

12. The method of claim 1, wherein said transfected cell population is allogeneic to the cancer patient in need of treatment.

13. The method of claim 1, wherein said transfected cell population is autologous to the cancer patient in need of treatment.

14. The method of claim 1, wherein an inhibitor of a CD3 inhibitory molecule is co-administered together with the CAR.

15. The method of claim 14, wherein said inhibitor of CD3 inhibitory molecule is a dominant negative CTLA-4.

16. The method of claim 14, wherein said inhibitor of CD3 inhibitory molecule is a dominant negative IL-10 receptor.

17. The method of claim 14, wherein said inhibitor of CD3 inhibitory molecule is a dominant negative TGF-beta receptor.

18. The method of claim 1, wherein said CAR transfected cells are cotransfected with an a molecule capable of inducing RNA interference.

19. The method of claim 18, wherein said molecule capable of inducing RNA interference are selected from a group comprising of:
a) siRNA; or
b) shRNA.

20. The method of claim 19, wherein silencing of molecules that inhibit CD3 zeta signaling are silenced.

21. The method of claim 20, wherein silencing of molecules is achieved, said molecules selected from a group comprising of:
a) OX2;
b) TGF-beta receptor;
c) SMAD4;
d) IL-10 receptor;
e) PD-1; and
f) CTLA-4.
